(19) United States
(12) Patent Application Publication
(10) Pub. No.: US 2017/0352695 A1
(43) Pub. Date: Dec. 7, 2017

(54) IMAGE ACQUISITION DEVICE, BIO-INFORMATION ACQUISITION DEVICE, AND ELECTRONIC APPARATUS

(71) Applicant: Seiko Epson Corporation, Tokyo (JP)

(72) Inventor: Hitoshi TSUCHIYA, Suwa (JP)

(21) Appl. No.: 15/536,043

(22) PCT Filed: Nov. 19, 2015
(86) PCT No.: PCT/JP2015/005798

§ 371 Date: Jun. 14, 2017

(30) Foreign Application Priority Data
Dec. 17, 2014 (JP) 2014-254827

(51) Int. Cl. H01L 27/146; A61B 5/1455; A61B 5/145; A61B 5/00

(52) U.S. Cl. CPC H01L 27/14623; H01L 27/14629; H01L 27/14627; A61B 5/0077; A61B 5/1455; A61B 5/14532; A61B 5/681

(57) ABSTRACT

An image acquisition device includes an imager including a light receiver, a light shield, a light condenser, and a light emitter. The light shield includes a light transmitting substrate, a light shielding layer, and an opening in the light shielding layer. A light transmitting layer having a refractive index smaller than that of the substrate is between the light condenser and the light shield. When a diameter of a light receiving surface of the light reception element is d, a diameter of the opening is a, a pitch of the light reception elements is p, a refractive index of the light transmitting layer is n1, a refractive index of the substrate is n2, and a distance between the light reception element and the light shielding layer is h, Arctan((p-a/2-d/2)/h)≧Arcsin(n1/n2).

### CROSS REFERENCE TO RELATED APPLICATIONS

This application is a U.S. National Phase Application under 35 U.S.C. 371 of International Application No. PCT/JP2015/005798 filed on Nov. 19, 2015 and published in Japanese as WO 2016/098283 A1 on Jun. 23, 2016. This application claims priority to Japanese Patent Application No. 2014-254827 filed Dec. 17, 2014. The entire disclosures of all of the above applications are incorporated herein by reference.

### TECHNICAL FIELD

The present invention relates to an image acquisition device, a bio-information acquisition device, and an electronic apparatus.

### BACKGROUND ART

An imaging device which images a subject and acquires an image is disclosed (JP-A-2014-67577). The imaging device disclosed in JP-A-2014-67577 has a structure in which a light receiving section, a light shielding section, a light emitting section, and a light condensing section are laminated sequentially. After incident light from the subject, which is illuminated by imaging light emitted from the light emitting section, is condensed by the light condensing section, the incident light passes through opening sections which are respectively provided in the light emitting section and the light shielding section, and reaches the light receiving section which is located in a bottom layer. The light receiving section includes a plurality of light reception elements, and is formed to perform image processing on intensity of the incident light, which is incident into the plurality of respective light reception elements, from the subject, thereby acquiring image information of the subject.

The light emitting section exemplified in the imaging device includes a first electrode layer, a second electrode layer, and a light emitting layer which is interposed between both the electrode layers and is formed by an organic Electro Luminescence (EL) material. A light emitting region in the light emitting section is prescribed by an insulating layer which is provided to surround a region in which the first electrode layer is in contact with the light emitting layer. In JP-A-2014-67577, an example is shown in which a location of the light emitting region for an optical axis of a lens is prescribed such that light, which is reflected on a surface of the lens as the light condensing section, among imaging light emitted from the light emitting section is not incident into light receiving surfaces of the light reception elements other than the incident light from the illuminated subject.

### SUMMARY OF INVENTION

### Technical Problem

However, in the imaging device disclosed in JP-A-2014-67577, the second electrode layer of the light emitting section is provided as a common electrode which is common to a plurality of first electrode layers, and includes parts which are provided other than the light emitting region and face the first electrode layers through the insulating layer. A surface of the first electrode layer has a light reflection property, and the insulating layer and the second electrode layer are formed by materials which have different refractive indexes. Therefore, there is a problem in that light emitted from the light emitting layer is reflected on, for example, the surface of the first electrode layer other than the light emitting region, and, furthermore, is reflected again on a boundary surface between the insulating layer and the second electrode layer, thereby generating so-called stray light. In a case where the stray light is incident into the light receiving surfaces of the light reception elements, there is a problem in that intensity of incident light from the subject is affected, and thus it is difficult to acquire a clear image of the subject. Meanwhile, the stray light includes not only the light which is reflected on the boundary surface of the insulating layer and the second electrode layer but also light which is refracted on a boundary surface of a member, through which light passes, from the light condensing section to the light receiving section.

### SOLUTION TO PROBLEM

The present invention has been made to solve at least a part of the above-described problems, and can be realized as embodiments or application examples below.

An image acquisition device according to this application example includes: an imaging section that includes a light reception element; a light shielding section; and a light emitting section that includes a light emitting element, in which the light shielding section includes a substrate that has a light transmitting property, a light shielding layer that is provided on a surface, which faces the imaging section, of the substrate, and an opening section that is provided in the light shielding layer so as to correspond to a disposition of the light reception element in the imaging section, in which a light transmitting layer, which has a refractive index smaller than a refractive index of the substrate of the light shielding section, is provided between the light emitting section and the light shielding section, and in which, in a case where a diameter of a light receiving surface of the light reception element is set to d, a diameter of the opening section is set to a, a disposition pitch of the light reception elements is set to p, a refractive index of the light transmitting layer is set to n1, the refractive index of the substrate is set to n2, and a distance between the light reception element and the light shielding layer is set to h, the following Expression is satisfied.

Arctan((p-a/2-d/2)/h)≧Arcsin(n1/n2)

According to the Snell laws, Arcsin(n1/n2) indicates a critical angle (hereinafter, referred to as a critical angle θm) of light which is incident into the light transmitting layer from the substrate of the light shielding section. In contrast, Arctan((p-a/2-d/2)/h) indicates an angle θ acquired in a case where light, which is incident from one opening section among opening sections that are adjacent in the light shielding section, is incident into the light receiving surface of the light reception element which faces another opening section. An incident angle of light, which is incident into the substrate of the light shielding section from the light transmitting layer, is refracted thereon, and is incident into the opening section of the light shielding section, is smaller than the critical angle θm. That is, in a case where a value of the angle θ is equal to or larger than the critical angle θm, light, which is incident into one opening section of the light shielding section, is not incident into the light receiving surface of the light reception element which faces another opening section.

According to this application example, it is possible to reduce the amount of stray light which is generated due to light emitted from the light emitting section and is incident into the light receiving surface of the light reception element from the opening section. Therefore, the amount of stray light, which is incident into the light receiving surface of the light reception element, is reduced, and thus it is possible to provide the image acquisition device which is capable of acquiring a clear image.

In the image acquisition device according to the application example, it is preferable that an adhesion layer is included between the imaging section and the light shielding section, and a refractive index n3 of the adhesion layer is approximately equal to the refractive index n2 of the substrate.

According to this configuration, the imaging section is strongly bonded to the light shielding section by the adhesion layer, and, even though the stray light is incident into the opening section, since it is difficult for an emission angle of the stray light from the opening section to be changed, it is difficult for the stray light to reach the light receiving surface of the light reception element. That is, it is possible to acquire a clear image and it is possible to provide the image acquisition device which has excellent durability.

The image acquisition device according to the application example may further include a light condensing section that includes a condensing lens which is disposed on an optical axis, in which the light reception element is connected with the opening section, between the light emitting section and the light shielding section, and the light transmitting layer may be provided between the light shielding section and the light condensing section.

According to this configuration, it is possible to condense incident light from a subject, which is illuminated by imaging light emitted from the light emitting section, into the light reception element by the condensing lens. In addition, compared to a case where the light condensing section is disposed on an upper side of the light emitting section, it is possible to prevent the stray light which is generated because light emitted from the light emitting section is reflected on the lens surface of the condensing lens. That is, it is possible to provide the image acquisition device which is capable of acquiring a further clear image.

In the image acquisition device according to the application example, it is preferable that the light transmitting layer is a vacuum layer or an air layer.

According to this configuration, the refractive index n1 of the light transmitting layer is approximately 1. Therefore, compared to a case where the refractive index n1 of the light transmitting layer is larger than 1, a refraction angle acquired in a case where the stray light is incident into the substrate of the light shielding section from the light transmitting layer becomes larger, and thus it is difficult for the stray light refracted on the substrate to be incident into the opening section of the light shielding section. That is, it is possible to provide the image acquisition device which is hardly affected by the stray light.

In the image acquisition device according to the application example, it is preferable that the light emitting element includes a reflecting layer that has a light reflection property, an electrode that has a light transmission property, and a light-emitting function layer that is disposed between the reflecting layer and the electrode, the light emitting section includes an insulating layer that is disposed between the reflecting layer and the electrode and decides a light emitting region in the light-emitting function layer, and a light transmitting section that is disposed between the adjacent light emitting elements, and an outer edge of the reflecting layer is located on a side of the light transmitting section rather than an end of the insulating layer on the side of the light transmitting section.

According to this configuration, it is possible to reflect the stray light, which is emitted from the light-emitting function layer of the light emitting element and has a possibility of being leaked to a side of the light transmitting section through the insulating layer, by the reflecting layer. That is, since it is difficult for the stray light to reach the imaging section, it is possible to acquire a further clear image.

A bio-information acquisition device according to this application example includes: an imaging section that includes a light reception element; a light shielding section; and a light emitting section that includes a light emitting element which emits near infrared light, in which the light shielding section includes a substrate that has a light transmitting property, a light shielding layer that is provided on a surface, which faces the imaging section, of the substrate, and an opening section that is provided in the light shielding layer so as to correspond to a disposition of the light reception element in the imaging section, in which a light transmitting layer, which has a refractive index smaller than a refractive index of the substrate of the light shielding section, is provided between the light emitting section and the light shielding section, and in which, in a case where a diameter of a light receiving surface of the light reception element is set to d, a diameter of the opening section is set to a, a disposition pitch of the light reception elements is set to p, a refractive index of the light transmitting layer is set to n1, the refractive index of the substrate is set to n2, and a distance between the light reception element and the light shielding layer is set to h, the following Expression is satisfied.

Arctan((p-a/2-d/2)/h)≧Arcsin(n1/n2)

According to the Snell laws, Arcsin(n1/n2) indicates a critical angle (hereinafter, referred to as a critical angle θm) of light which is incident into the light transmitting layer from the substrate of the light shielding section. In contrast, Arctan((p-a/2-d/2)/h) indicates an angle θ acquired in a case where light, which is incident from one opening section among opening sections that are adjacent in the light shielding section, is incident into the light receiving surface of the light reception element which faces another opening section. An incident angle of light, which is incident into the substrate of the light shielding section from the light transmitting layer, is refracted thereon, and is incident into the opening sections of the light shielding section, is smaller than the critical angle θm. That is, in a case where a value of the angle θ is equal to or larger than the critical angle θm, light, which is incident into one opening section of the light shielding section, is not incident into the light receiving surface of the light reception element which faces another opening section.

According to this application example, it is possible to reduce the amount of stray light which is generated due to light (near infrared light) emitted from the light emitting section and is incident into the light receiving surface of the light reception element from the opening section. Therefore, the amount of stray light, which is incident into the light receiving surface of the light reception element, is reduced, and thus it is possible to provide the bio-information acquisition device which is capable of acquiring clear bio-information.

In the bio-information acquisition device according to the application example, it is preferable that an adhesion layer is included between the imaging section and the light shielding section, and a refractive index n3 of the adhesion layer is approximately equal to the refractive index n2 of the substrate.

According to this configuration, the imaging section is strongly bonded to the light shielding section by the adhesion layer, and, even though the stray light is incident into the opening section, since it is difficult for an emission angle of the stray light from the opening section to be changed, it is difficult for the stray light to reach the light receiving surface of the light reception element. That is, it is possible to acquire clear bio-information and it is possible to provide the bio-information acquisition device which has excellent durability.

The bio-information acquisition device according to the application example may further include a light condensing section that includes a condensing lens which is disposed on an optical axis, in which the light reception element is connected with the opening section, between the light emitting section and the light shielding section, and the light transmitting layer may be provided between the light shielding section and the light condensing section.

According to this configuration, it is possible to condense incident light from a subject, which is illuminated by imaging light emitted from the light emitting section, into the light reception element by the condensing lens. In addition, compared to a case where the light condensing section is disposed on an upper side of the light emitting section, it is possible to prevent the stray light which is generated because light emitted from the light emitting section is reflected on the lens surface of the condensing lens. That is, it is possible to provide the bio-information acquisition device which is capable of acquiring further clear bio-information.

In the bio-information acquisition device according to the application example, it is preferable that the light transmitting layer is a vacuum layer or an air layer.

According to this configuration, the refractive index n1 of the light transmitting layer is approximately 1. Therefore, compared to a case where the refractive index n1 of the light transmitting layer is larger than 1, a refraction angle acquired in a case where the stray light is incident into the substrate of the light shielding section from the light transmitting layer becomes larger, and thus it is difficult for the stray light refracted on the substrate to be incident into the opening section of the light shielding section. That is, it is possible to provide the bio-information acquisition device which is hardly affected by the stray light.

In the bio-information acquisition device according to the application example, it is preferable that the light emitting element includes a reflecting layer that has a light reflection property, an electrode that has a light transmission property, and a light-emitting function layer that is disposed between the reflecting layer and the electrode, the light emitting section includes an insulating layer that is disposed between the reflecting layer and the electrode and decides a light emitting region in the light-emitting function layer, and a light transmitting section that is disposed between the adjacent light emitting elements, and an outer edge of the reflecting layer is located on a side of the light transmitting section rather than an end of the insulating layer on the side of the light transmitting section.

According to this configuration, it is possible to reflect the stray light, which is emitted from the light-emitting function layer of the light emitting element and has a possibility of being leaked to a side of the light transmitting section through the insulating layer, by the reflecting layer. That is, since it is difficult for the stray light to reach the imaging section, it is possible to acquire further clear bio-information.

An electronic apparatus according to this application example includes the image acquisition device according to the above application example.

According to this application example, it is possible to provide the electronic apparatus which is capable of acquiring a clear image. For example, in a case where an image, such as a face or fingerprint of an operator, is acquired by the image acquisition device, it is possible to provide an information terminal device as the electronic apparatus which ensures security of the operator.

An electronic apparatus according to this application example includes the bio-information acquisition device according to the above application example.

According to this application example, it is possible to provide the electronic apparatus which is capable of acquiring clear bio-information. For example, in a case where blood component information, such as a blood-sugar level of an examinee, is acquired by the bio-information acquisition device, it is possible to provide an electronic apparatus which is capable of performing health management of the examinee.

### BRIEF DESCRIPTION OF DRAWINGS

FIG. 1 is a perspective view illustrating a configuration of a portable information terminal as an electronic apparatus.

FIG. 2 is a block diagram illustrating an electrical configuration of the portable information terminal.

FIG. 3 is a perspective view schematically illustrating a configuration of a sensor section.

FIG. 4 is a sectional view schematically illustrating a structure of the sensor section.

FIG. 5 is a sectional view typically illustrating a configuration of a light emitting element.

FIGS. 6(a) and (b) are plan views schematically illustrating disposition of light emitting elements, light transmitting sections, and light reception elements.

FIG. 7 is a sectional view schematically illustrating a structure of a light emitting section.

FIG. 8 is a sectional view schematically illustrating structures of a light condensing section, a light shielding section, and an imaging section in the sensor section.

FIG. 9 is a sectional view schematically illustrating a structure of a sensor section as a bio-information acquisition device according to a second embodiment.

FIG. 10 is a plan view schematically illustrating disposition of light emitting elements and light reception elements in an image acquisition device according to a third embodiment.

FIG. 11 is a sectional view schematically illustrating a structure of light emitting elements according to a modification example.

### DESCRIPTION OF EMBODIMENTS

Hereinafter, embodiments which embody the present invention will be described with reference to the accompanying drawings. Meanwhile, drawings to be used are displayed by being appropriately enlarged or reduced such that parts to be described become recognizable states.

### First Embodiment

### <Electronic Apparatus>

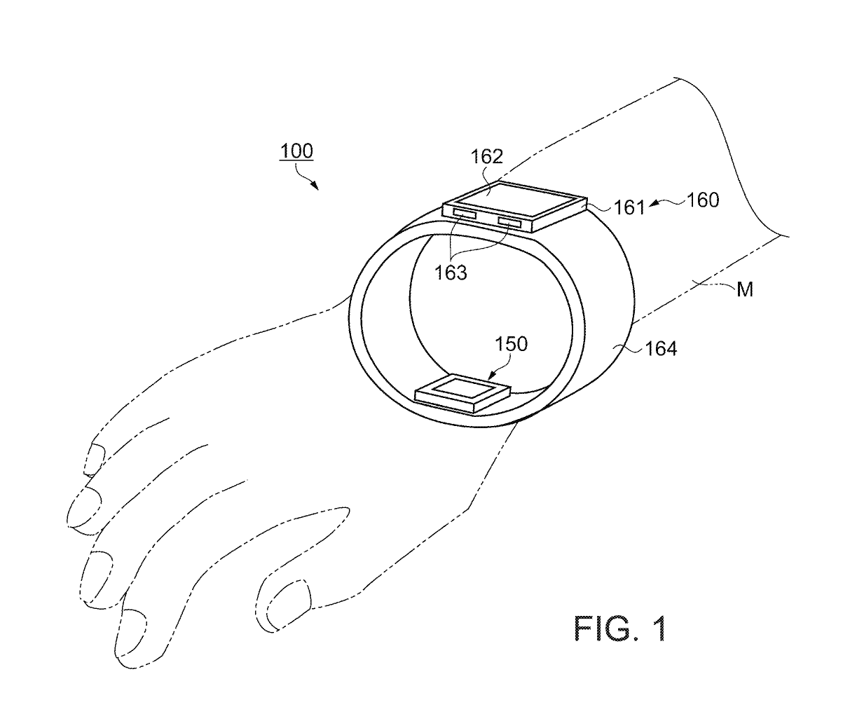

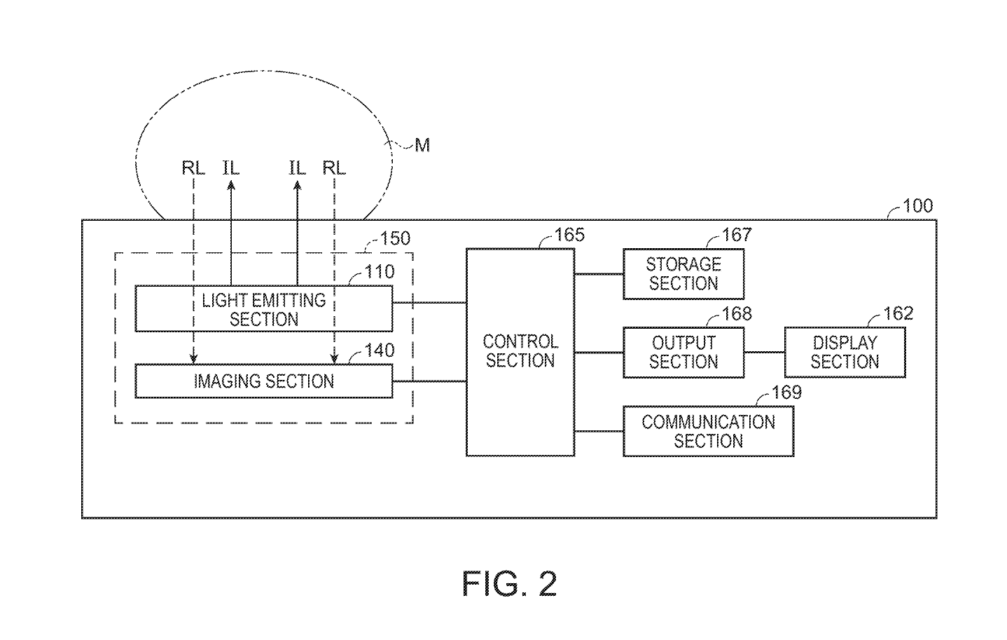

First, an electronic apparatus according to an embodiment will be described using a portable information terminal as an example with reference to FIGS. 1 and 2. FIG. 1 is a perspective view illustrating a configuration of the portable information terminal as the electronic apparatus, and FIG. 2 is a block diagram illustrating an electrical configuration of the portable information terminal as the electronic apparatus.

As illustrated in FIG. 1, a portable information terminal 100 as the electronic apparatus according to the embodiment is a device which is mounted on a wrist of a human body M and is capable of obtaining information such as an image of a blood vessel inside the wrist or a specific component in blood of the blood vessel. The portable information terminal 100 includes a circular belt 164 that is attachable to the wrist, a main body section 160 that is attached to the outside of the belt 164, and a sensor section 150 that is attached to the inside of the belt 164 in a location which faces the main body section 160. The main body section 160 includes a main body case 161 and a display section 162 that is incorporated in the main body case 161. In addition to the display section 162, operational buttons 163, a circuit system (see FIG. 2), such as a control section 165 which will be described later, a battery as a power supply, and the like are incorporated in the main body case 161.

The sensor section 150 is an example of a bio-information acquisition device according to the present invention, and is electrically connected to the main body section 160 through wirings (not shown in FIG. 1) incorporated in the belt 164. It is preferable that the belt 164 has elasticity when taking a mounting property on the human body M into consideration.

The portable information terminal 100 is used by being mounted on the wrist such that the sensor section 150 is in contact with the wrist on a palm side which is opposite to the back of the hand. In a case where the portable information terminal 100 is mounted in this manner, it is possible to prevent a detection sensitivity of the sensor section 150 from being changed depending on a skin color.

Meanwhile, although the portable information terminal 100 according to the embodiment is configured such that the main body section 160 and the sensor section 150 are separately incorporated in the belt 164, the portable information terminal 100 may be configured such that the main body section 160 and the sensor section 150 are integrally incorporated in the belt 164.

As illustrated in FIG. 2, the portable information terminal 100 includes a control section 165, the sensor section 150 which is electrically connected to the control section 165, a storage section 167, an output section 168, and a communication section 169. In addition, the portable information terminal 100 further includes the display section 162 that is electrically connected to the output section 168.

The sensor section 150 includes a light emitting section 110 and an imaging section 140. The light emitting section 110 and the imaging section 140 are electrically connected to the control section 165, respectively. The light emitting section 110 includes light emitting elements that emit near infrared light IL which has a wavelength in a range of 700 nm to 2000 nm. The control section 165 drives the light emitting section 110 and causes the light emitting section 110 to emit the near infrared light IL. The near infrared light IL is propagated and scattered inside the human body M. It is configured such that it is possible to receive a part of the near infrared light IL scattered inside the human body M as reflected light RL by the imaging section 140.

The control section 165 is capable of storing information of the reflected light RL received by the imaging section 140 in the storage section 167. In addition, the control section 165 causes the output section 168 to process the information of the reflected light RL. The output section 168 converts the information of the reflected light RL into information of an image of the blood vessel and outputs the resulting information or converts the information of the reflected light RL into information of a specific component included in blood and outputs the resulting information. In addition, the control section 165 is capable of displaying the information of the image of the blood vessel and the information of the specific component in blood, which are acquired through conversion, on the display section 162. In addition, it is possible to transmit the pieces of information from the communication section 169 to another information processing device. In addition, the control section 165 is capable of receiving information, such as a program, from another information processing device through the communication section 169, and storing the received information in the storage section 167. The communication section 169 may be a wired communication section that is connected to another information processing device by a wired line, and may be a wireless communication section such as Bluetooth (registered trademark). Meanwhile, the control section 165 may not only display the acquired information related to the blood vessel and blood on the display section 162 but also display information of a program, which is stored in the storage section 167 in advance, and information of current time, and the like on the display section 162. In addition, the storage section 167 may be a detachable memory.

### <Bio-Information Acquisition Device>

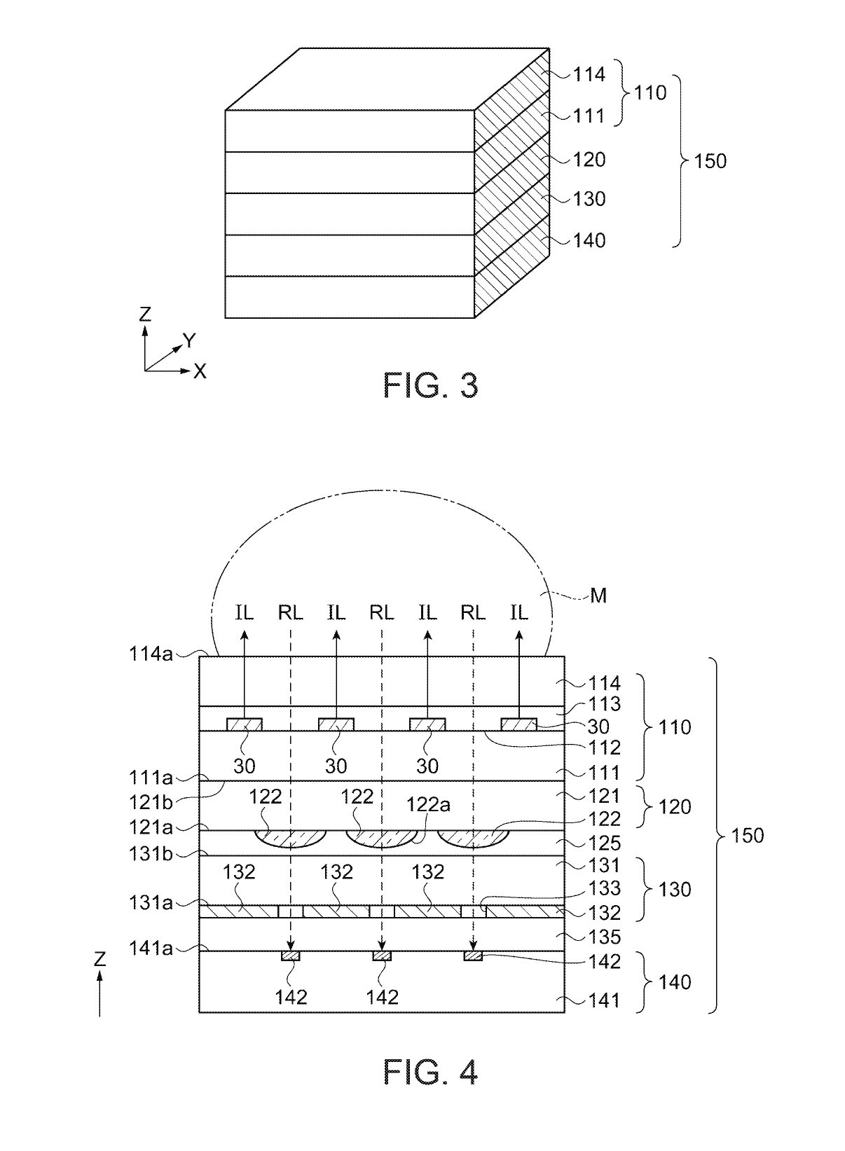

Subsequently, the sensor section 150 as the bio-information acquisition device according to the embodiment will be described with reference to FIGS. 3 and 4. FIG. 3 is a perspective view schematically illustrating a configuration of the sensor section, and FIG. 4 is a sectional view schematically illustrating a structure of the sensor section.

As illustrated in FIG. 3, the sensor section 150 includes the light emitting section 110, a light condensing section 120, a light shielding section 130, and the imaging section 140. The respective sections have plate shapes, respectively, and are configured such that the light shielding section 130, the light condensing section 120, and the light emitting section 110 are sequentially laminated on the imaging section 140. The sensor section 150 receives a laminated body in which the respective sections are laminated, and includes a case (not shown in the drawing) which is attachable to the belt 164 of the portable information terminal 100. Meanwhile, the light emitting section 110 includes an element substrate 111 in which light emitting elements are formed, a protective substrate 114 which protects the light emitting element. Hereinafter, description will be performed while a direction along one side of the laminated body is set to an X direction, a direction along another side which is perpendicular to the one side is set to a Y direction, and a thickness direction of the laminated body is set to a Z direction. In addition, viewing from a side of the protective substrate 114 along the Z direction is referred to as a planar view.

As illustrated in FIG. 4, the light emitting section 110 is formed to include the element substrate 111 in which light emitting elements 30 are provided, a sealing layer 113 that seals the light emitting elements 30 such that moisture or the like does not permeate the light emitting elements 30, and the protective substrate 114 that is disposed to face the element substrate 111 through the sealing layer 113.

The protective substrate 114 is, for example, a cover glass or plastic substrate which has a light transmitting property. The human body M is disposed to be in contact with one surface 114a of the protective substrate 114. Hereinafter, the substrate which has the light transmitting property indicates a substrate which is formed of glass or plastics, and the light transmitting property indicates that transmittance is at least equal to or higher than 85% in a representative wavelength of light emitted from the light emitting section 110.

The sealing layer 113 is formed of, for example, a thermosetting epoxy resin or an acrylic resin, and has the light transmitting property.

The substrate which has the light transmitting property is also used as a substrate main body of the element substrate 111. Although details will be described later, the light emitting elements 30 are formed to emit near infrared light IL in the element substrate 111 to a side of the protective substrate 114 and are capable of illuminating the human body M disposed on the protective substrate 114. The element substrate 111 includes the light transmitting sections 112 that guide the reflected light RL, which is reflected from the inside the illuminated human body M and is incident into the light emitting section 110, to the light condensing section 120 on the lower layer. The light transmitting sections 112 are disposed between the light emitting elements 30 which are disposed to be adjacent.

The light condensing section 120 includes a substrate 121 which has the light transmitting property, and a plurality of condensing lenses 122 that are provided on one surface 121a of the substrate 121. The light condensing section 120 and the light emitting section 110 are bonded such that projected lens surfaces 122a of the condensing lenses 122 face the light shielding section 130. In addition, the light condensing section 120 and the light emitting section 110 are bonded such that optical centers of the condensing lenses 122 are located on the optical axis of the reflected light RL which passes through the light emitting section 110. In other words, a disposition gap between the light transmitting sections 112 in the light emitting section 110 is basically the same as a disposition gap between the condensing lenses 122 in the light condensing section 120.

The light shielding section 130 includes a substrate 131 which has the light transmitting property, and a light shielding layer 132 that is provided on a surface 131a of the substrate 131 on a side opposite to a surface 131b on the side of the light condensing section 120. In the light shielding layer 132, opening sections (pinholes) 133 are formed in locations corresponding to the dispositions of the light transmitting sections 112 of the light emitting section 110. The light shielding section 130 is disposed between the light condensing section 120 and the imaging section 140 such that only the reflected light RL, which passes through the opening sections 133, is led to light reception elements 142, and remaining reflected light RL is shielded by the light shielding layer 132. The light shielding layer 132 is formed using, for example, a metal film, which has a light shading property and is formed by a metal, such as Cr, or an alloy thereof, or a resin film which includes a light absorbing material capable of absorbing at least near infrared light.

The light condensing section 120 and the light shielding section 130 are disposed to face each other through a light transmitting layer 125. Specifically, the light transmitting layer 125 is an empty space and is formed by a vacuum layer or an air layer. In other words, the surface 121a, on which the condensing lenses 122 of the light condensing section 120 are provided, and the surface 131b of the light shielding section 130 are disposed to face each other with a prescribed gap, and the light condensing section 120 is bonded to the light shielding section 130 under vacuum or under atmospheric pressure.

The imaging section 140 is an image sensor for near infrared light, and includes a substrate 141 and a plurality of light reception elements 142 that are provided on a surface 141a of the substrate 141 on a side of the light shielding section 130. It is possible to use, for example, an optical sensor, such as a CCD or a CMOS, as the light reception element 142. The substrate 141 may include, for example, a glass epoxy substrate or a ceramic substrate, on which it is possible to mount the light reception elements 142, or a semiconductor substrate, in which it is possible to vertically form the light reception elements 142 thereon, and includes an electric circuit (not shown in the drawing) to which the light reception elements 142 are connected. The plurality of light reception elements 142 are disposed on the surface 141a of the substrate 141 in locations corresponding to the dispositions of the opening sections 133 of the light shielding section 130.

It is known that optical sensors, which are used as the light reception elements 142, have different sensitivities according to wavelengths. For example, a CMOS sensor has higher sensitivity for visible light than sensitivity for near infrared light IL. In a case where the CMOS sensor receives visible light in addition to near infrared light IL (reflected light RL), visible light is output from the CMOS sensor as noise. Therefore, for example, filters that cut light in a visible light wavelength range (400 nm to 700 nm) may be disposed to correspond to the light transmitting sections 112 of the light emitting section 110 or the opening section 133 of the light shielding section 130.

The light shielding section 130 and the imaging section 140 are disposed to face each other with a prescribed gap, and are boned to each other through an adhesion layer 135 which has the light transmitting property. In the embodiment, respective members, which form the substrate 131 and the adhesion layer 135, are selected such that a refractive index of the substrate 131 of the light shielding section 130 is almost equal to a refractive index of the adhesion layer 135. For example, the substrate 131 of the light shielding section 130 is a quartz glass substrate (refractive index n2≈1.53), and the adhesion layer 135 is an epoxy-based resin (refractive index n3≈1.55).

Meanwhile, the configuration of the sensor section 150 is not limited thereto. For example, the light emitting section 110 may include a structure which seals the light emitting elements 30 by the protective substrate 114 without the sealing layer 113. In addition, since there is a problem in that the reflected light RL, which passes through the light transmitting sections 112, is attenuated by being reflected on a boundary surface of a member, through which the reflected light passes, it is preferable that the light emitting section 110 is bonded to the light condensing section 120 such that, for example, a surface 111a of the element substrate 111 of the light emitting section 110 on a side of the light condensing section 120 is in contact with a surface 121b of the substrate 121 of the light condensing section 120 on a side of the light emitting section 110. In addition, in this manner, it is possible to ensure a locational relationship between the light transmitting sections 112 and the condensing lenses 122 in the thickness direction (Z direction).

### [Light Emitting Element]

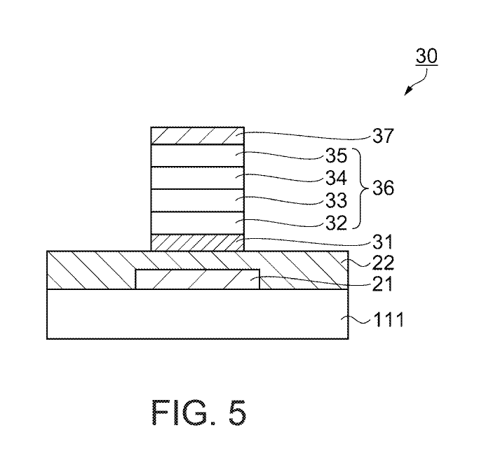

Subsequently, the light emitting element 30 will be described with reference to FIG. 5. FIG. 5 is a sectional view typically illustrating a configuration of the light emitting element.

As illustrated in FIG. 5, the light emitting element 30 includes a reflecting layer 21 that is provided on the element substrate 111 and has a light reflection property, an anode 31 that has a light transmission property, a light-emitting function layer 36, and a cathode 37 that functions as an electrode which has the light transmission property. An interlayer insulation film 22 that adjusts a distance between the reflecting layer 21 and the anode 31 is provided between the reflecting layer 21 and the anode 31. The light-emitting function layer 36 includes a hole injection transport layer 32, a light emitting layer 33, an electron transport layer 34, and an electron injection layer 35 which are sequentially laminated from a side of the anode 31. In the light emitting element 30, holes injected from the side of the anode 31 are recombined with electrons injected from a side of the cathode 37 in the light emitting layer 33, and thus energy, emitted in a case of the recombination, is emitted as light. The light emitting layer 33 includes a light emitting material which is formed of an organic semiconductor material, and the light emitting element 30 is referred to as an organic electro-luminescence (EL) element. Light emitted from the light emitting layer 33 is emitted after passing through the cathode 37. In addition, a part of the emitted light passes through the anode 31 and is reflected on the reflecting layer 21, passes through the anode 31 again, and is emitted from the side of the cathode 37. That is, it is possible to extract almost light emitted in the light emitting layer 33 from the side of the cathode 37. The light emitting element 30 is referred to as a top emission type.

### [Reflecting Layer]

It is possible to form the reflecting layer 21 using, for example, a metal, such as Al (aluminum) or Ag (silver), which has a light reflection property, or an alloy thereof. In a case where the light reflection property and productivity are taken into consideration, it is preferable that a combination of Al (aluminum) and Cu (copper), a combination of Al (aluminum) and Nd (neodymium), or the like is used as the alloy. A film thickness of the reflecting layer 21 is set to, for example, 200 nm by taking the light reflection property into consideration.

### [Anode]

The anode 31 is formed using, for example, a transparent conductive film, such as ITO, which has a large work function by taking a hole injection property into consideration. A film thickness of the anode 31 is set to, for example, 15 nm by taking a light transmission property into consideration.

### [Cathode]

The cathode 37 is formed to have the light reflection property and the light transmission property by controlling film thickness using, for example, an alloy including Ag and Mg. A film thickness of the cathode 37 is, for example, 20 nm. Meanwhile, the cathode 37 is not limited to an alloy layer formed of Ag and Mg, and may have, for example, a multi-layered structure in which a layer formed of Mg is laminated on the alloy layer formed of Ag and Mg. With the configuration which includes the reflecting layer 21, the anode 31, and the cathode 37, a part of light emitted from the light-emitting function layer 36 of the light emitting element 30 is repeatedly reflected between the cathode 37 and the reflecting layer 21, and is emitted after an intensity of light having a specific wavelength is enhanced based on an optical distance between the cathode 37 and the reflecting layer 21. That is, an optical resonant structure in which the intensity of light having a specific wavelength is enhanced is introduced for the light emitting element 30. The interlayer insulation film 22, which is provided between the reflecting layer 21 and the anode 31, is provided to adjust the optical distance in the optical resonant structure, and is formed using, for example, a silicon oxide.

### [Light Emitting Layer]

The light emitting layer 33 of the light-emitting function layer 36 includes a light emitting material (organic semiconductor material) in which light emitted in a near-infrared wavelength range (700 nm to 20000 nm) is acquired. It is possible to give, for example, a well-known light emitting material, such as a thiadiazole-based compound or a selenadiazole-based compound, as the light emitting material. In addition, in addition to the light emitting material, a host material in which the light emitting material is added (supported) as a guest material (dopant) is used. The host material has functions of generating exciton by recombining holes and electrons, moving (Foerster movement or Dexter movement) exciton energy to the light emitting material, and exciting the light emitting material. Therefore, it is possible to increase light-emitting efficiency. For example, a light emitting material, which is the guest material, is doped as the dopant and is used for the host material.

Particularly, it is preferable that a quinolinolato metal complex or an acene-based organic compound is used as the host material. An anthracene-based material or a tetracene-based material is preferable in the acene-based material, and the tetracene-based material is further preferable. In a case where the host material of the light emitting layer 33 is formed to include the acene-based material, it is possible to effectively deliver electrons from an electron transport material in the electron transport layer 34, which will be described later, to the acene-based material in the light emitting layer 33.

In addition, the acene-based material has excellent resistance for the electrons and the holes. In addition, the acene-based material has excellent thermal stability. Therefore, it is possible to realize the long-life light emitting element 30. In addition, since the acene-based material has the excellent thermal stability, it is possible to prevent the host material from being decomposed by heat generated when a film is formed in a case where the light emitting layer 33 is formed using a vapor phase film deposition method. Therefore, it is possible to form the light emitting layer 33 which has excellent film quality. As a result, at this point, it is possible to increase light-emitting efficiency of the light emitting element 30 and to realize the long life.

Furthermore, since it is difficult for the acene-based material to emit light in itself, it is possible to prevent the host material from adversely affecting a light emission spectrum of the light emitting element 30.

It is preferable that a light emitting material content (doping amount) in the light emitting layer 33, which includes the light emitting material and the host material, is in a range of 0.01 wt % to 10 wt %, and a range of 0.1 wt % to 5 wt % is further preferable. In a case where the light emitting material content is included in the range, it is possible to optimize the light-emitting efficiency.

In addition, although an average thickness of the light emitting layer 33 is not particularly limited, it is preferable that the average thickness of the light emitting layer 33 is in a degree of 1 nm to 60 nm, and a degree of 3 nm to 50 nm is further preferable.

### [Hole Injection Transport Layer]

The hole injection transport layer 32 is formed to include a hole injection transport material for improving a hole injection property and a hole transport property for the light emitting layer 33. It is possible to exemplify, for example, an aromatic amine compound, in which a part of a framework is selected among a phenylenediamine system, a benzidine system, and a terphenylenediamine system, as the hole injection transport material.

Although the average thickness of the hole injection transport layer 32 is not particularly limited, it is preferable that the average thickness of the hole injection transport layer 32 is in a degree of 5 nm to 200 nm, and a degree of 10 nm to 100 nm is further preferable.

Meanwhile, in the light emitting element 30, a layer which is provided between the anode 31 and the light emitting layer 33 is not limited to only the hole injection transport layer 32. For example, a plurality of layers that include a hole injection layer, into which holes are easily injected from the anode 31, and a hole transport layer, in which holes are easily transported to the light emitting layer 33, may be provided. In addition, a layer, which blocks electrons that are leaked from the light emitting layer 33 to the side of the anode 31, may be included.

### [Electron Transport Layer]

The electron transport layer 34 has a function of transporting electrons injected from the cathode 37 through the electron injection layer 35 to the light emitting layer 33. As a material (electron transport material) which forms the electron transport layer 34, for example, a phenanthroline derivative such as 2,9-dimethyl-4, 7-diphenyl-1, or 10-phenanthroline (BCP), a quinoline derivative such as 8-quinolinol including tris(8-quinolinolato) aluminum (Alq3) or an organic metal complex in which the derivative is used as a ligand, an azaindolizine derivative, an oxadiazole derivative, a perylene derivative, a pyridine derivative, a pyrimidine derivative, a quinoxaline derivative, a diphenyl quinone derivative, a nitro substituted fluorene derivative, and the like are exemplified. It is possible to combine one or more types of the above derivatives and to use a resulting derivative.

In addition, in a case where two or more types of the above-described electron transport materials are combined to use the resulting material, the electron transport layer 34 may be formed of a mixture material in which two or more types of electron transport materials are mixed, or may be formed by laminating a plurality of layers which are formed by different electron transport materials.

Particularly, in a case where a tetracene derivative is used as the host material in the light emitting layer 33, it is preferable that the electron transport layer 34 includes an azaindolizine derivative. The azaindolizine derivative, which has an anthracene framework in a molecule, is further preferable. It is possible to effectively deliver electrons from the anthracene framework in the azaindolizine derivative molecule to the host material.

Although the average thickness of electron transport layer 34 is not particularly limited, it is preferable that the average thickness is in a degree of 1 nm to 200 nm, and a degree of 10 nm to 100 nm is further preferable.

Meanwhile, a layer provided between the light emitting layer 33 and the electron injection layer 35 is not limited to only the electron transport layer 34. For example, a plurality of layers may be provided that include a layer in which it is easy to inject electrons from the electron injection layer 35, a layer in which it is easy to transport electrons to the light emitting layer 33, and a layer which is used to control the amount of electrons to be injected to the light emitting layer 33. In addition, a layer may be included that has a function of blocking holes which are leaked to a side of the electron injection layer 35 from the light emitting layer 33.

### [Electron Injection Layer]

The electron injection layer 35 has a function of improving electron injection efficiency from the cathode 37.

For example, various inorganic insulating materials and various inorganic semiconductor materials are exemplified as component materials (materials having an electron injection property) of the electron injection layer 35.

For example, alkaline metal chalcogenide (an oxide, a sulfide, a selenide, and a telluride), alkaline-earth metal chalcogenide, alkaline metal halogenide, and alkaline-earth metal halogenide, and the like are exemplified as the inorganic insulating materials, and it is possible to combine one or more types of inorganic insulating materials and to use the resulting material. In a case where the electron injection layer (EIL) is formed using the inorganic insulating materials as main materials, it is possible to improve the electron injection property. Particularly, an alkaline metal compound (alkaline metal chalcogenide, alkaline metal halogenide, and the like) has an extremely small work function, and, in a case where the electron injection layer 35 is formed using the compound, the light emitting element 30 may provide high light-emitting brightness.

For example, Li₂O, LiO, Na₂S, Na₂Se, NaO, and the like are exemplified as the alkaline metal chalcogenide.

For example, CaO, BaO, SrO, BeO, BaS, MgO, CaSe, and the like are exemplified as the alkaline-earth metal chalcogenide.

For example, CsF, LiF, NaF, KF, LiCl, KCl, NaCl, and the like are exemplified as the alkaline metal halogenide.

For example, CaF₂, BaF₂, SrF₂, MgF₂, BeF₂, and the like are exemplified as the alkaline-earth metal halogenide.

In addition, for example, an oxide, a nitride, an oxynitride, or the like which includes at least one element among Li, Na, Ba, Ca, Sr, Yb, Al, Ga, In, Cd, Mg, Si, Ta, Sb, and Zn, is exemplified as the inorganic semiconductor material, and it is possible to combine one or more types of materials and to use the resulting material.

Although the average thickness of the electron injection layer 35 is not particularly limited, a degree of 0.1 nm to 1000 nm is preferable, a degree of 0.2 nm to 100 nm is further preferable, and a degree of 0.2 nm to 50 nm is further preferable.

Meanwhile, the electron injection layer 35 may be omitted depending on the component materials or the thickness of the cathode 37 and the electron transport layer 34.

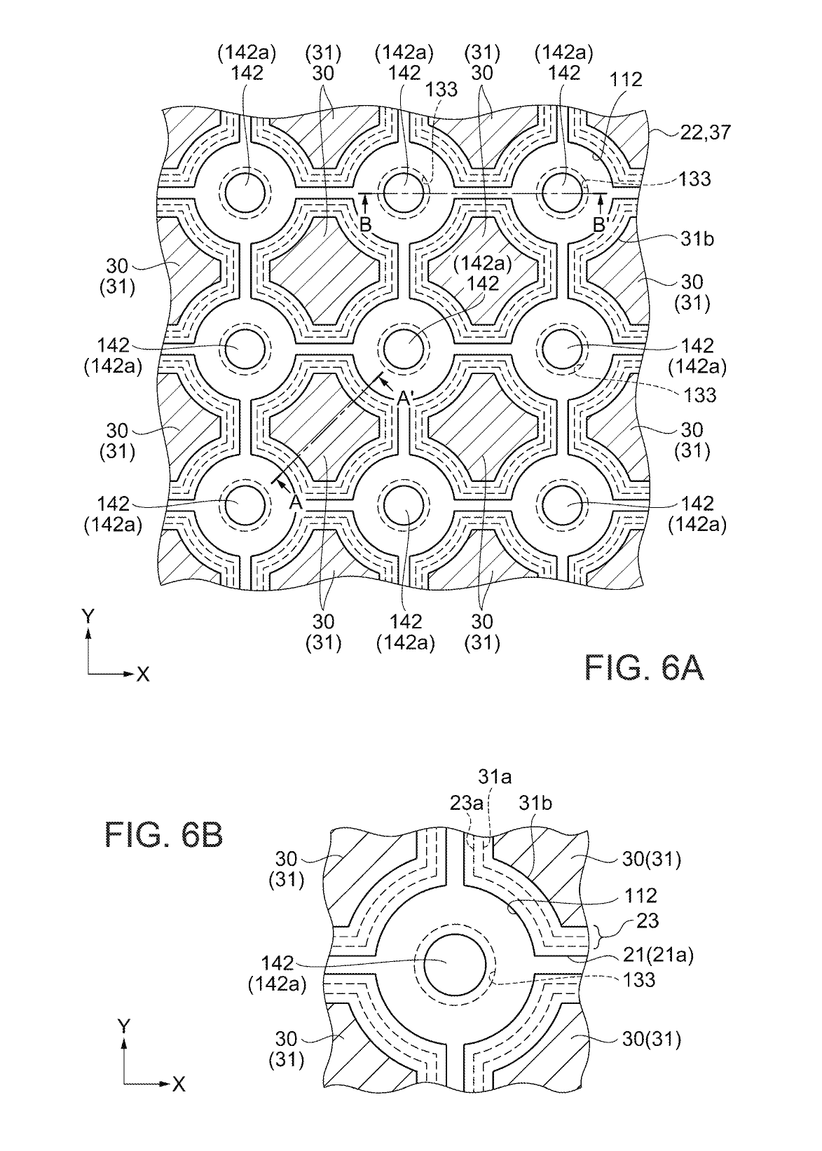

Subsequently, a disposition relationship between the light emitting elements 30, the light transmitting sections 112, and the light reception elements 142 in the sensor section 150 will be described with reference to FIGS. 6(a) and 6(b). FIGS. 6(a) and 6(b) are plan views schematically illustrating disposition of the light emitting elements, the light transmitting sections, and the light reception elements.

As illustrated in FIGS. 6(a) and 6(b), the light reception elements 142, to which the reflected light RL from the human body M is led, are disposed in a matrix shape with prescribed gaps in the X direction and the Y direction. Light receiving surfaces 142a of the light reception elements 142 are formed in circle shapes. The light transmitting sections 112, which lead the reflected light RL to the light reception elements 142, are formed in approximately circle shapes around the light reception elements 142 such that the reflected light RL is uniformly and evenly led to the light receiving surfaces 142a. The opening sections 133 of the light shielding sections 130 are disposed around the light reception elements 142 on inner sides of the light transmitting sections 112, and are formed in circle shapes which are larger than the light receiving surfaces 142a.

Therefore, the planar shapes of the light emitting elements 30 disposed between the light transmitting sections 112 are formed in approximately rhombic shapes which are surrounded by arcs. The planar shapes of the light emitting elements 30 are prescribed by shapes of the reflecting layers 21, the anodes 31, and partition wall sections 23. Specifically, the approximately circular light transmitting sections 112 are prescribed by arc-shaped parts of outer edges 21a of the approximately rhombic-shaped reflecting layers 21. The anodes 31, which are disposed inner sides of the reflecting layers 21 in a planar view, have sizes slightly smaller than the reflecting layers 21, and have approximately rhombic shapes similarly to the reflecting layers 21. The partition wall sections 23 corresponding to insulating layers according to the present invention are provided to overlap outer edges 31a of the anodes 31, and prescribes regions in which the anodes 31 are in contact with the light-emitting function layers 36, that is, light emitting regions 31b of the light emitting elements 30. Therefore, planar shapes of the light emitting regions 31b are slightly smaller than the anodes 31 and have approximately rhombic shapes.

The reflecting layers 21 and the anodes 31 are independently provided for the plurality of respective light emitting elements 30. In contrast, the interlayer insulation films 22, which cover the reflecting layers 21, are provided across the plurality of reflecting layers 21. In addition, the cathodes 37 are provided as common electrodes across the plurality of light emitting elements 30.

As described above, the sensor section 150 according to the embodiment includes the plurality of light emitting elements 30 and the plurality of light reception elements 142, and is in a state in which four light emitting elements 30 are disposed in the vicinity of one light reception element 142 (light transmitting section 112). In other words, the sensor section 150 is in a state in which four light reception elements 142 (light transmitting sections 112) are disposed in the vicinity of one light emitting element 30. It is preferable that the number of light reception elements 142, which are disposed in a matrix shape in the X direction and the Y direction, in the imaging section 140 is equal to or larger than, for example, 240×240=57600, from a viewpoint in which bio-information is acquired with high accuracy.

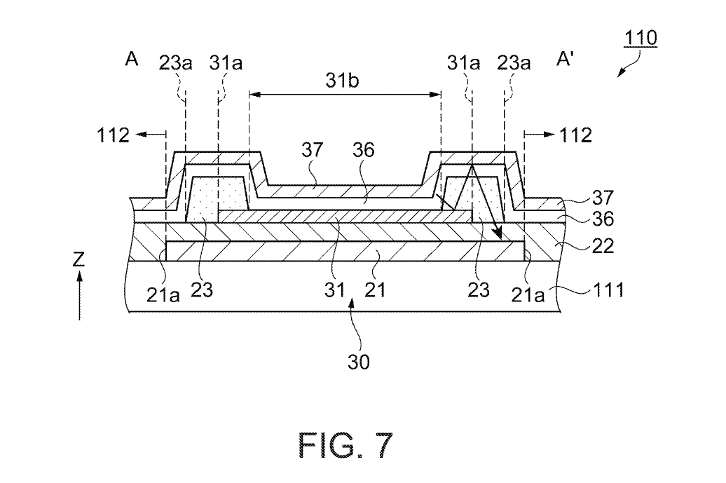

Subsequently, a detailed structure of the light emitting section 110 will be described with reference to FIG. 7. FIG. 7 is a sectional view schematically illustrating the structure of the light emitting section. Specifically, FIG. 7 is a sectional view schematically illustrating the structures of the light emitting element 30 and the light transmitting section 112 taken along a line A-A′ which passes through the reflecting layer 21 illustrated in FIG. 6(a) in a direction of an angle of 45°.

As illustrated in FIG. 7, the light emitting section 110 includes the light emitting element 30 and the light transmitting section 112 which are formed on the element substrate 111. On the element substrate 111, first, a film, which is formed of, for example, a metal, such as Al (aluminum), that has the light reflection property or an alloy thereof, is formed, and the reflecting layer 21 is formed by patterning the film. Subsequently, the interlayer insulation film 22, which covers the reflecting layer 21 over the entire surface of the element substrate 111, is formed. For example, a transparent conductive film, such as ITO, is formed on the interlayer insulation film 22, and the anode 31 is formed on an upper side of the reflecting layer 21 by patterning the transparent conductive film. The outer edge 31a of the anode 31 is patterned to be located on an inner side than the outer edge 21a of the reflecting layer 21. The partition wall section 23 is formed in a location which overlaps the outer edge 31a of the anode 31. It is possible to form the partition wall section 23 as the insulating layer using an inorganic or organic insulating material. In the embodiment, a photosensitive resin film, which has a film thickness of 1.0 μm to 2.0 μm, is formed over the approximately entire surface of the element substrate 111. The partition wall section 23 is formed by patterning the photosensitive resin film. The partition wall section 23 is patterned to surround the light emitting region 31b in which the anode 31 is in contact with the light-emitting function layer 36. In addition, the partition wall section 23 is patterned such that an end 23a of the partition wall section 23 on a side opposite to the light emitting region 31b is located between the outer edge 21a of the reflecting layer 21 and the outer edge 31a of the anode 31. Subsequently, the light-emitting function layer 36 is formed over the approximately entire surface of the element substrate 111 on which the partition wall section 23 is formed. As described above, the light-emitting function layer 36 includes the hole injection transport layer 32, the light emitting layer 33, the electron transport layer 34, and the electron injection layer 35, and the respective layers are formed to be sequentially laminated using, for example, the vapor phase film deposition method such as a vacuum evaporation method. The respective layers are not limited to the layers which are formed using the vapor phase film deposition method, and a part of the layers may be formed using a liquid phase deposition method. Subsequently, the cathode 37, which covers the light-emitting function layer 36 over the approximately entire surface of the element substrate 111, is formed to have the light reflection property and the light transmission property using an alloy of Ag and Mg by, for example, the vapor phase film deposition method such as the vacuum evaporation method.

As described above, the light emitting element 30 includes the reflecting layer 21, the interlayer insulation film 22, the anode 31, the light-emitting function layer 36, and the cathode 37. The light transmitting section 112, which is formed between the light emitting elements 30 on the element substrate 111, includes the interlayer insulation film 22, the light-emitting function layer 36, and the cathode 37. Meanwhile, although not illustrated in FIG. 7, a pixel circuit, which is capable of applying an electrical current between the anode 31 and the cathode 37 by performing electrical switching control on the anode 31 of the light emitting element 30, is provided between the substrate main body of the element substrate 111 and the reflecting layer 21. The pixel circuit includes transistors and storage capacities as switching elements, and wirings which connect them. The reflecting layer 21 functions as a relay electrode which applies an electrical potential to the anode 31 by the pixel circuit.

According to the structure of the light emitting section 110, the most of light emitted from the light emitting region 31b of the top emission-type light emitting element 30 is emitted from the side of the cathode 37. In contrast, there is a problem in that, at a part where the partition wall section 23 is provided on the outside of the light emitting region 31b, light emitted from the light-emitting function layer 36 is reflected on a surface of the anode 31 as illustrated by a solid line arrow of FIG. 7, thereafter, is reflected on the boundary between the light-emitting function layer 36 and the cathode 37, and is leaked to the outside from the outer edge 31a of the anode 31. However, since the reflecting layer 21 is disposed on the outside from the outer edge 31a of the anode 31, the leaked light (stray light) is reflected by the reflecting layer 21. That is, since the stray light, which is leaked through the partition wall section 23, is reflected on the reflecting layer 21 as described above, a structure is provided in which it is difficult for the stray light to be incident into the light transmitting section 112 between the light emitting elements 30.

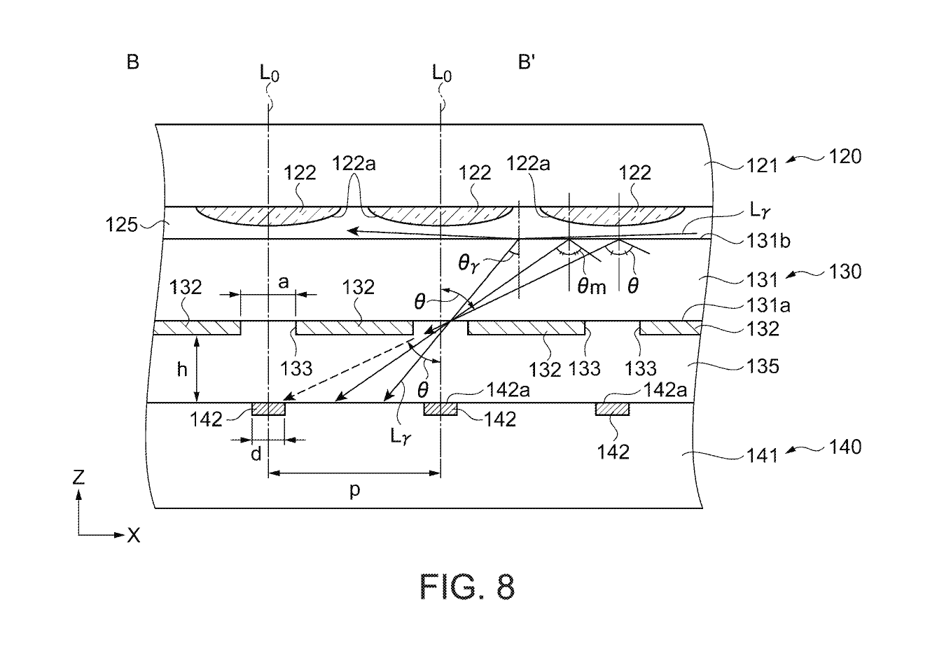

Subsequently, the detailed structures of the light condensing section 120, the light shielding section 130, and the imaging section 140 will be described with reference to FIG. 8. FIG. 8 is a sectional view schematically illustrating the structures of the light condensing section, the light shielding section, and the imaging section in the sensor section. Specifically, FIG. 8 is a sectional view schematically illustrating the structures of the light condensing section 120, the light shielding section 130, and the imaging section 140 taken along a line B-B′ which is illustrated in FIG. 6(a) and crosses the light reception elements 142 which are adjacent in the X direction. Also, for easy understanding of description, refraction angles of an optical axis are exaggeratedly drawn in FIG. 8.

As illustrated in FIG. 8, the light shielding section 130 is laminated on the imaging section 140 through the adhesion layer 135, and, further, the light condensing section 120 is laminated on the light shielding section 130 through the light transmitting layer 125. Since the light transmitting layer 125 is a vacuum layer or an air layer as described above, there is a case where the light transmitting layer 125 is referred to as an empty space 125. On an optical axis L₀, which passes through a center of the condensing lens 122 having the projected lens surface 122a, a center of the light receiving surface 142a of the light reception elements 142 and a center of the opening section 133 of the light shielding layer 132 are located. Meanwhile, actually, in a case where the imaging section 140, the light shielding section 130, and the light condensing section 120 are laminated, the center of the condensing lenses 122, the center of the light receiving surface 142a of the light reception elements 142, and the center of the opening section 133 of the light shielding layer 132 may be located for the optical axis L₀ in an allowance range of a manufacturing process in an in-plane which is vertical to the optical axis L₀.

As described above, the reflected light RL, which is emitted from the human body M illuminated by the light emitting section 110, is incident into the condensing lens 122 of the light condensing section 120. The reflected light RL, which is condensed by the condensing lens 122, passes through the opening section 133 of the light shielding section 130 and is incident into the light reception element 142 of the imaging section 140. In other words, relative locations of the condensing lens 122, the opening section 133, and the light reception element 142 on the optical axis L₀ are determined by taking a focal distance of the condensing lens 122 into consideration such that the reflected light RL, which is condensed by the condensing lens 122, is incident into the light reception element 142.

In contrast, light, which is incident into another surface 131b that faces one surface 131a on which the light shielding layer 132 of the substrate 131 is provided, includes both the reflected light RL which is condensed by the condensing lens 122 and the reflected light RL which is not incident into the condensing lens 122. Since the empty space 125, which has a smaller refractive index than the refractive index of the substrate 131, exists between the light condensing section 120 and the substrate 131 of the light shielding section 130, light which is incident into another surface 131b of the substrate 131 from a side of the empty space 125 is refracted by the substrate 131. However, it is not limited that the whole refracted light is incident into the light reception element 142.

For example, as illustrated by a solid line arrow in FIG. 8, there is a possibility in that, in one light reception element 142 and another light reception element 142 which are disposed to be adjacent in the X direction in the imaging section 140, light, which is incident into the opening section 133 that faces another light reception element 142, is incident into one light reception element 142. The light is treated also as the stray light which affects the reflected light RL that is incident into one light reception element 142. In the embodiment, a size of the opening section 133 for a size of the light receiving surface 142a of the light reception element 142 and a relative locational relationship between the light reception element 142 and the opening section 133 are prescribed such that it is difficult for the stray light to be incident into one light reception element 142.

Specifically, in a case where a diameter of the light receiving surface 142a of the light reception element 142 is set to “d”, a diameter of the opening section 133 is set to “a”, a disposition pitch of the light reception element 142 is set to “p”, a refractive index of the empty space (light transmitting layer) 125 is set to “n1”, a refractive index of the substrate 131 is set to “n2”, and a distance between the light reception element 142 and the light shielding layer 132 is set to “h”, respective values of the diameter d, the diameter a, the disposition pitch p, and the distance h are prescribed such that the following Expression (1) is satisfied.

Arctan((p-a/2-d/2)/h)≧Arcsin(n1/n2) . . . (1)

According to the Snell laws, θm=Arcsin(n1/n2) indicates a critical angle θm, as illustrated in FIG. 8, in a case where light heads to the empty space 125 which has the refractive index n1 from the substrate 131 which has the refractive index n2 of the light shielding section 130. In contrast, θ=Arctan((p-a/2-d/2)/h) indicates an angle θ in a case where light, which is incident from one opening section 133 (opening section 133 which is drawn at the center in FIG. 8) among the opening sections 133 that are adjacent in the light shielding section 130, is incident into the light receiving surface 142a of the light reception element 142 which faces another opening section 133 (opening section 133 which is drawn on the left side in FIG. 8). An incident angle θγ of light Lγ, which is incident into the substrate 131 from the empty space 125, is refracted, and is incident into the opening section 133 of the light shielding section 130, is smaller than the critical angle θm. That is, in a case where the incident angle θγ is slightly smaller than the critical angle θm, an optical path in which light enters the substrate 131 from a side of the empty space 125 exists as an optical path of light Lγ which is incident into the opening section 133. In a case where the incident angle θγ is equal to the critical angle θm, a total reflection condition is satisfied, and thus an optical path which enters the substrate 131 from the side of the empty space 125 does not exist. However, in a case where a virtual optical path is taken into consideration, the virtual optical path is parallel to another surface 131b of the substrate 131. In this manner, in a case where the value of the angle θ is equal to or larger than the critical angle θm, light, which is incident into one opening section 133 of the light shielding section 130, is not incident into the light receiving surface 142a of the light reception elements 142 which faces another opening section 133. Meanwhile, in the embodiment, the refractive index n2 of the substrate 131 is approximately equal to the refractive index n3 of the adhesion layer 135 as described above. Therefore, in a case where an incident angle of light L3 which is incident into the opening section 133 is the angle θ, an incident angle of light which is incident into the light receiving surface 142a of the light reception elements 142 from the opening section 133 becomes almost the same angle θ.

In the embodiment, for example, the diameter d of the light receiving surface 142a of the light reception elements 142 is 10 μm, the diameter a of the opening section 133 is 16 μm, the distance h between the light reception elements 142 and the light shielding layer 132 is 100 μm, the disposition pitch p of the light reception elements 142 in the X direction is 100 μm, the refractive index n1 of the empty space 125 is 1.0, and the refractive index n2 of the substrate 131 is approximately 1.53. Therefore, according to Expression (1), the critical angle θm≈40.8 and the angle θ≈41.0, and thus the amount of stray light, which affects the reflected light RL that is incident into the light reception elements 142, is reduced. Meanwhile, in the embodiment, since the empty space 125 is the vacuum layer or the air layer, it is assumed that the refractive index n1 is 1.0. However, the empty space 125, that is, the light transmitting layer 125 is not limited to the empty space. In a case where the light transmitting layer 125 is a layer which is formed of a material which has the light transmitting property and in which a value of the refractive index n1 is smaller than the refractive index n2 of the substrate 131, it is possible to specify the critical angle θm.

According to the sensor section 150 of the first embodiment, the amount of stray light, which is generated due to light (near infrared light) emitted from the light emitting section 110 and is incident into the light receiving surface 142a of the light reception elements 142 from the opening section 133, is reduced. Therefore, the reflected light RL, which is incident into the light receiving surface 142a, is hardly affected by the stray light, and thus it is possible to realize the sensor section 150 which is capable of acquiring clear bio-information.

In addition, according to the portable information terminal 100 as an electronic apparatus which includes the sensor section 150, it is possible to acquire pieces of information, such as an image of a blood vessel of the human body M on which the portable information terminal 100 is mounted and specific component in blood of the blood vessel, with high accuracy. For example, since influence of the stray light is reduced, it is possible to accurately obtain a change in light absorbance due to a change in concentration of the specific component in blood, thereby leading highly-accurate quantitative evaluation of the specific component.

Meanwhile, as illustrated in FIG. 7, the stray light includes light which is leaked to the side of the light transmitting section 112 through the partition wall section 23 located in the vicinity of the light emitting region 31b while the human body M is not irradiated with the near infrared light IL emitted from the light emitting elements 30. In addition, as illustrated in FIG. 8, the stray light includes light, which is incident into one light reception elements 142 from the opening section 133 that faces another light reception elements 142, in one light reception element 142 and another light reception element 142 which are disposed to be adjacent in the X direction. In addition, in FIG. 8, the light reception elements 142, which are adjacent in the X direction, and the opening section 133 are exemplified, light reception elements 142, which are adjacent in the Y direction, and the opening section 133 have the same relationship.

### Second Embodiment

### <Bio-Information Acquisition Device>

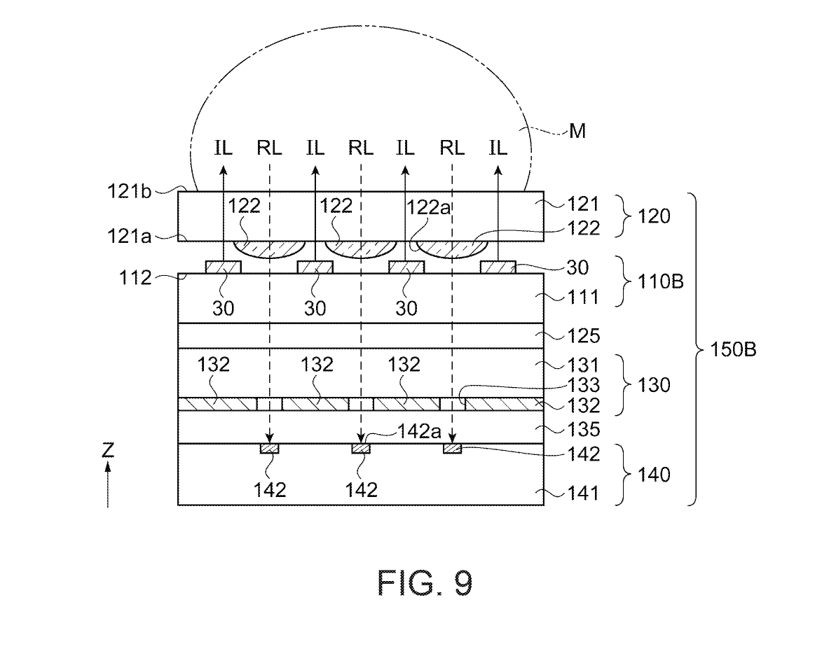

Subsequently, a bio-information acquisition device according to a second embodiment will be described with reference to FIG. 9. FIG. 9 is a sectional view schematically illustrating a structure of a sensor section as the bio-information acquisition device according to the second embodiment. A sensor section 150B as the bio-information acquisition device according to the second embodiment is different from the sensor section 150 according to the first embodiment in a configuration of the light emitting section 110 and disposition of the light condensing section 120. Therefore, the same reference symbols are attached to the same components as in the sensor section 150 according to the first embodiment and the detailed description thereof will not be repeated.

As illustrated in FIG. 9, the sensor section 150B as the bio-information acquisition device according to the embodiment includes the light condensing section 120, a light emitting section 110B, the light shielding section 130, and the imaging section 140. The respective sections have plate shapes, respectively, and are configured such that the light shielding section 130, the light emitting section 110B, and the light condensing section 120 are sequentially laminated on the imaging section 140. The sensor section 150B receives a laminated body in which the respective sections are laminated, and includes a case (not shown in the drawing) which is attachable to a belt 164 of the portable information terminal 100 as the electronic apparatus described in the first embodiment.

The light emitting section 110B includes the light emitting elements 30 and the element substrate 111 on which the light transmitting sections 112 are formed. In the embodiment, the light condensing section 120 functions as a protective substrate that protects the light emitting elements 30. Respective configurations on the element substrate 111 and the dispositions thereof are described with reference to FIGS. 5 and 7 in the first embodiment.

The light transmitting layer 125 is provided between the light emitting section 110B and the light shielding section 130. The light transmitting layer 125 is an empty space that has a prescribed thickness in the Z direction, and the empty space is a vacuum layer or an air layer. Therefore, in the embodiment, the light transmitting layer 125 is also referred to as the empty space 125.

The light shielding section 130 is bonded to the imaging section 140 through the adhesion layer 135. In the sensor section 150B, respective sections are laminated such that the center of the opening section 133 which is formed in the light shielding layer 132 of the light shielding section 130 and the center of the light receiving surface 142a of the light reception element 142 are located on an optical axis which passes through a center of a condensing lens 122 of the light condensing section 120.

The relationship, which is acquired in the diameter d of the light receiving surface 142a of the light reception element 142 in the imaging section 140, the disposition pitch p between the light reception elements 142, the diameter a of the opening section 133 in the light shielding section 130, the distance h between the light reception element 142 and the light shielding layer 132, the refractive index n1 of the empty space 125, the refractive index n2 of the substrate 131 of the light shielding section 130, satisfies Expression (1) in the first embodiment.

A human body M is disposed on the surface 121b which faces the surface 121a on which the condensing lenses 122 of the light condensing section 120 are provided. The human body M is illuminated by near infrared light IL emitted from the light emitting elements 30 of the light emitting section 110B, and reflected light RL which is reflected inside the illuminated human body M is incident into the light condensing section 120. The reflected light RL which is incident into the light condensing section 120 is condensed by the condensing lenses 122, passes through the light transmitting sections 112 of the element substrate 111, and is led to the light reception elements 142 of the imaging section 140.

The sensor section 150B outputs an image signal based on intensity of the reflected light RL which is incident into the plurality of light reception elements 142 in the imaging section 140.

According to the sensor section 150B of the second embodiment, it is possible to reduce the amount of stray light, which is generated due to light (near infrared light) emitted from the light emitting section 110B and is incident into the light receiving surface 142a of the light reception elements 142 from the opening section 133, similarly to the sensor section 150 according to the first embodiment. Therefore, it is difficult for the reflected light RL, which is incident into the light receiving surface 142a of the light reception elements 142, to be affected by the stray light, and thus it is possible to realize the sensor section 150B which is capable of acquiring clear bio-information.

Particularly, even though the light, which is emitted from the light emitting elements 30, is reflected on the lens surfaces 122a of the condensing lenses 122 and stray light, which is incident into the light transmitting sections 112, is generated in a case where the light condensing section 120 is disposed on an upper side of the light emitting section 110B, since the respective configurations in the light shielding section 130 and the imaging section 140 satisfy the above-described Expression (1), it is difficult for the stray light to be incident into the light reception elements 142. In addition, since it is possible to cause the light condensing section 120 to function as the protective substrate, it is possible to make a thickness of the sensor section 150B, which is a laminated body, be thin, compared to the sensor section 150.

Therefore, in a case where the sensor section 150B is included in the portable information terminal 100 as the electronic apparatus, it is possible to acquire an image of a blood vessel of the human body M, on which the sensor section 150B is mounted, and information of a specific component in blood of the blood vessel with high accuracy, and it is possible to realize a thin and lightweight portable information terminal 100.

### Third Embodiment

### <Image Acquisition Device>

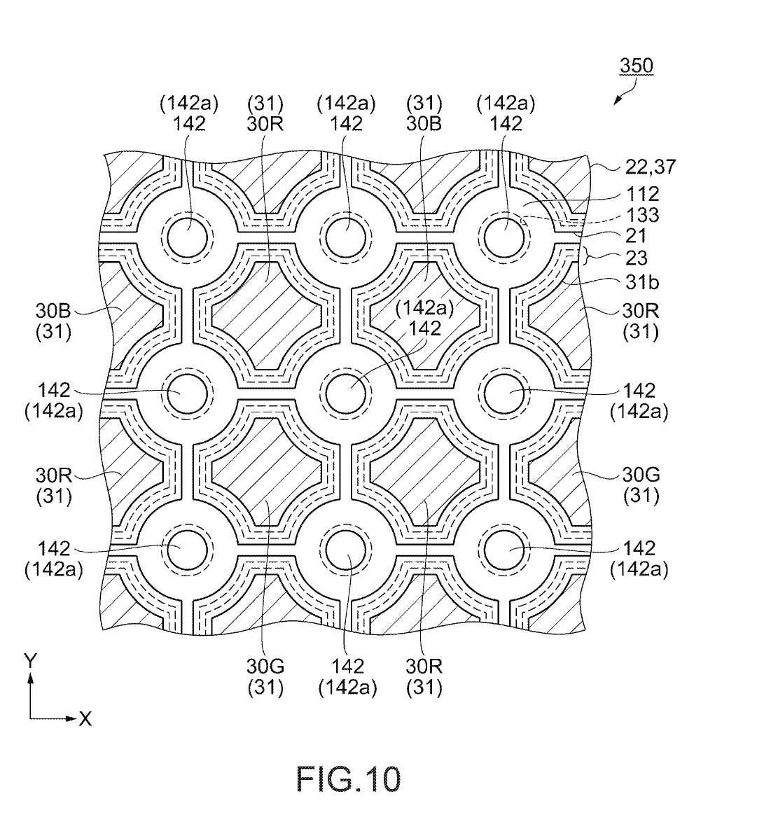

Subsequently, an image acquisition device according to a third embodiment will be described with reference to FIG. 10. FIG. 10 is a plan view schematically illustrating disposition of light emitting elements and light reception elements in the image acquisition device according to the third embodiment. An image acquisition device 350 according to the third embodiment is different from the sensor section 150 as the bio-information acquisition device according to the first embodiment in the configuration of the light emitting section 110. Therefore, the same reference symbols are attached to the same components as in the sensor section 150, and detailed description thereof will not be repeated.

The image acquisition device 350 according to the embodiment includes the light emitting section 110, the light condensing section 120, the light shielding section 130, and the imaging section 140, similarly to the sensor section 150 according to the first embodiment. The respective sections have plate shapes, respectively, and are configured such that the light shielding section 130, the light condensing section 120, and the light emitting section 110 are sequentially laminated on the imaging section 140. Meanwhile, a basic configuration of the image acquisition device 350 may be the same as that of the sensor section 150B according to the second embodiment. That is, the image acquisition device 350 may be a laminated body in which the light shielding section 130, the light emitting section 110, and the light condensing section 120 are sequentially laminated on the imaging section 140. In the embodiment, the configuration of the light emitting section 110 is different from the first embodiment, and thus the light emitting section 110 is referred to as a light emitting section 110C.

As illustrated in FIG. 10, the image acquisition device 350 includes the light reception elements 142 which are disposed in the X direction and the Y direction with prescribed gaps in the imaging section 140. In addition, the image acquisition device 350 includes approximately circular light transmitting sections 112 around the light reception elements 142 in a planar view, and three types of light emitting elements 30R, 30G, and 30B which are disposed between the light transmitting sections 112 that are located in the X direction and the Y direction with prescribed gaps in the light emitting section 110C.

All the light emitting elements 30R, 30G, and 30B are organic EL elements, emitted red color (R) light is acquired from the light emitting element 30R, emitted green color (G) light is acquired from the light emitting element 30G, and emitted blue color (B) light is acquired from the light emitting element 30B.

In addition, an element row, in which the light emitting element 30R and the light emitting element 30G are alternately disposed in the X direction, and an element row, in which the light emitting element 30B and the light emitting element 30R are alternately disposed in the X direction, are alternately disposed in the Y direction. Therefore, an element column, in which the light emitting element 30R and the light emitting element 30B are alternately disposed in the Y direction, and an element column, in which the light emitting element 30G and the light emitting element 30R are alternately disposed in the Y direction, are completed. That is, a state is provided in which one light emitting element 30B, one light emitting element 30G, and two light emitting elements 30R are disposed, respectively, around one light reception element 142 (light transmitting section 112). Meanwhile, dispositions of the three types of light emitting elements 30R, 30G, and 30B are not limited thereto. In addition, a light emitting element, from which emitted color light other than red (R), green (G), and blue (B) is acquired, may be disposed.

The configuration of the reflecting layer 21, the anode 31, the partition wall section 23, the cathode 37, and the like in each of the light emitting elements 30R, 30G, and 30B is basically the same as in the light emitting elements 30 according to the first embodiment, and light which is leaked from the partition wall section 23 on the outside of the light emitting region 31b is reflected on the reflecting layer 21 and is not incident into the side of the light transmitting section 112. In addition, the relationship, which is acquired in the diameter d of the light receiving surface 142a of the light reception elements 142 in the imaging section 140, the disposition pitch p of the light reception elements 142, the diameter a of the opening section 133 in the light shielding section 130, the distance h between the light reception element 142 and the light shielding layer 132, the refractive index n1 of the empty space 125, the refractive index n2 of the substrate 131 of the light shielding section 130, satisfies Expression (1) in the first embodiment.

Meanwhile, it is preferable that the film thickness of the interlayer insulation film 22 disposed between the reflecting layer 21 and the anode 31 is set for each of the light emitting elements 30R, 30G, and 30B which have different specific wavelengths from a viewpoint of enhancing intensity of light having the specific wavelength in an optical resonant structure.

According to the image acquisition device 350 of the third embodiment, it is possible to reduce the amount of stray light, which is generated due to light emitted from the light emitting section 110C and is incident into the light receiving surface 142a of the light reception elements 142 from the opening section 133. Therefore, it becomes difficult for the reflected light, which is incident into the light receiving surface 142a of the light reception elements 142 from the subject illuminated by the light emitting section 110C, to be affected by the stray light, and it is possible to realize the image acquisition device 350 which is capable of acquiring a clear image. In addition, since the light emitting section 110C includes the three types of light emitting elements 30R, 30G, and 30B, it is possible to acquire a color image of the subject. In addition, since it is possible to independently control light emission of the respective light emitting elements 30R, 30G, and 30B, it is possible to acquire an image according to a state of the subject.

In a case where the image acquisition device 350 is replaced by, for example, the sensor section 150 in the portable information terminal 100 according to the first embodiment and a finger is imaged as a subject, it is possible to acquire fingerprint information. In a case where the acquired fingerprint information is used, it is possible to perform security management for identifying an operating person. In addition, for example, in a case where influence of the stray light is reduced, it is possible to accurately obtain a change in light absorbance (three wavelengths) due to a change in concentration of the specific component in blood, thereby leading highly-accurate quantitative evaluation of the specific component.

The present invention is not limited to the above-described embodiment, appropriate change is possible in a range which does not depart from claims and the gist or the spirit of the invention which is read throughout the specification, and an image acquisition device and a bio-information acquisition device, which involve the change, and an electronic apparatus, to which the image acquisition device and the bio-information acquisition device are applied, are included in a technical range of the present invention. In addition to the embodiments, various modification examples are conceivable. Hereinafter, description will be performed by exemplifying modification examples.

### Modification Example 1

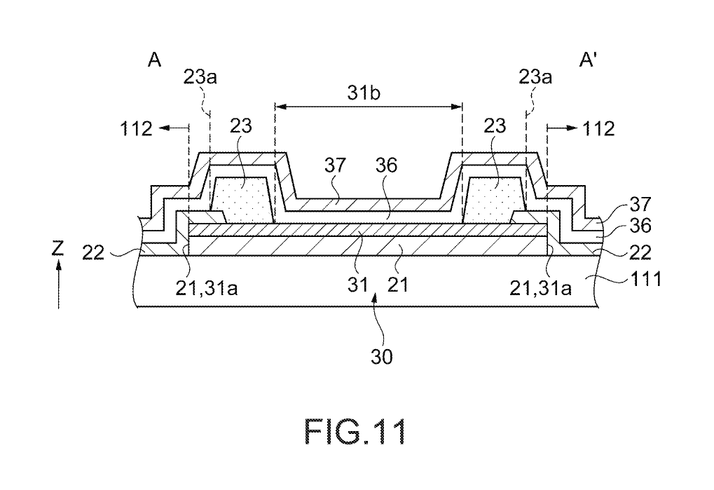

The present invention is not limited to the configuration in which the interlayer insulation film 22 disposed between the reflecting layer 21 and the anode 31 in the light emitting element 30 according to the first embodiment. FIG. 11 is a sectional view schematically illustrating a structure of the light emitting element according to a modification example. Specifically, FIG. 11 is a sectional view schematically illustrating the light emitting element taken along a line A-A′ of FIG. 6(a), similarly to FIG. 7 according to the first embodiment.

As illustrated in FIG. 11, the light emitting element 30 according to the modification example includes the anode 31 that is directly laminated on the reflecting layer 21 having the light reflection property and that has a light transmission property. The interlayer insulation film 22 is formed such that the outer edges 21a and 31a of the reflecting layer 21 and the anode 31 are covered and at least the light emitting region 31b is exposed in the anode 31. The partition wall section 23 is formed such that the light emitting region 31b is enclosed on the anode 31 and a part thereof overlaps the interlayer insulation film 22. The end 23a on the side of the light transmitting section 112 of the partition wall section 23 is located between the outer edge of the light emitting region 31b and the outer edges 21a and 31a of the reflecting layer 21 and the anode 31. According to the structure of the light emitting element 30 of the modification example, it is possible to reflect light, which is leaked to the side of the light transmitting section 112, by the reflecting layer 21 through the partition wall section 23 which is located in the vicinity of the light emitting region 31b, similarly to the first embodiment. In addition, it is possible to electrically and easily connect the reflecting layer 21 to the anode 31.

### Modification Example 2

In each embodiment, the planar shape of the light emitting region 31b is not limited to the approximately rhombic shape. For example, the planar shape of the light emitting region 31b may be a circular shape or a polygon such as a rectangular shape.

### Modification Example 3

In each embodiment, the reflecting layer 21 is not limited to the reflecting layer which is independently provided for each light emitting element. For example, the reflecting layer 21 may be formed over the plurality of light emitting elements 30, and the light transmitting section 112 may be formed in a circular shape by removing a part of the reflecting layer 21, which overlaps the light reception element 142 in a planar view. In this case, the reflecting layer 21 is electrically separated from the anode 31.

### Modification Example 4

The image acquisition device 350 according to the third embodiment is not limited to the three types of light emitting elements 30R, 30G, and 30B included in the light emitting section 110C. For example, a configuration may be provided in which one or two types of light emitting elements that are capable of emitting light in a visible light wavelength region are included. Furthermore, a configuration may be provided which includes a light emitting element that emits light in the visible light wavelength region and a light emitting element that emits light in a near-infrared wavelength region. Accordingly, it is possible to acquire image information of the subject and internal bio-information of the subject.

### Modification Example 5

The electronic apparatus, to which the sensor section 150 or the sensor section 150B as the bio-information acquisition device is applied, is not limited to the portable information terminal 100. For example, in a case where any one of the sensor sections 150 and 150B is applied to a personal computer, it is possible to perform biometric authentication which specifies a user of the personal computer from the image of the blood vessel. In addition, it is possible to acquire information of a specific component in blood of the user.

In addition, for example, it is possible to apply the present invention to a device, which measures blood pressure, blood sugar, a pulse, a pulse wave, the amount of cholesterol, the amount of hemoglobin, blood water, the amount of oxygen in the blood, and the like, as a medical instrument. In addition, it is possible to measure a liver function (detoxification rate), to check a blood vessel location, and to check a cancer part by using together with pigment. Furthermore, it is possible to determine a benign malignant tumor (melanoma) of a skin cancer by extending knowledge in specimens. In addition, in a case where a part or the whole items are comprehensively determined, it is possible to determine a skin age and an index of a health level of the skin.

1. An image acquisition device comprising:
an imaging section that includes a light reception element;
a light shielding section; and
a light emitting section that includes a light emitting element,
wherein the light shielding section includes a substrate that has a light transmitting property, a light shielding layer that is provided on a surface, which faces the imaging section, of the substrate, and an opening section that is provided in the light shielding layer so as to correspond to a disposition of the light reception element in the imaging section,
wherein a light transmitting layer, which has a refractive index smaller than a refractive index of the substrate of the light shielding section, is provided between the light emitting section and the light shielding section, and
wherein, in a case where a diameter of a light receiving surface of the light reception element is set to d, a diameter of the opening section is set to a, a disposition pitch of the light reception elements is set to p, a refractive index of the light transmitting layer is set to n1, the refractive index of the substrate is set to n2, and a distance between the light reception element and the light shielding layer is set to h, the following Expression is satisfied.
Arctan((p-a/2-d/2)/h)≧Arcsin(n1/n2)

2. The image acquisition device according to claim 1,
wherein an adhesion layer is included between the imaging section and the light shielding section, and
wherein a refractive index n3 of the adhesion layer is approximately equal to the refractive index n2 of the substrate.

3. The image acquisition device according to claim 1, further comprising:
a light condensing section that includes a condensing lens which is disposed on an optical axis, in which the light reception element is connected with the opening section, between the light emitting section and the light shielding section,
wherein the light transmitting layer is provided between the light shielding section and the light condensing section.

4. The image acquisition device according to claim 1,
wherein the light transmitting layer is a vacuum layer or an air layer.

5. The image acquisition device according to claim 1,
wherein the light emitting element includes a reflecting layer that has a light reflection property, an electrode that has a light transmission property, and a light-emitting function layer that is disposed between the reflecting layer and the electrode,
wherein the light emitting section includes an insulating layer that is disposed between the reflecting layer and the electrode and decides a light emitting region in the light-emitting function layer, and a light transmitting section that is disposed between the adjacent light emitting elements, and
wherein an outer edge of the reflecting layer is located on a side of the light transmitting section rather than an end of the insulating layer on the side of the light transmitting section.

6. A bio-information acquisition device comprising:
an imaging section that includes a light reception element;
a light shielding section; and
a light emitting section that includes a light emitting element which emits near infrared light,
wherein the light shielding section includes a substrate that has a light transmitting property, a light shielding layer that is provided on a surface, which faces the imaging section, of the substrate, and an opening section that is provided in the light shielding layer so as to correspond to a disposition of the light reception element in the imaging section,
wherein a light transmitting layer, which has a refractive index smaller than a refractive index of the substrate of the light shielding section, is provided between the light emitting section and the light shielding section, and
wherein, in a case where a diameter of a light receiving surface of the light reception element is set to d, a diameter of the opening section is set to a, a disposition pitch of the light reception elements is set to p, a refractive index of the light transmitting layer is set to n1, the refractive index of the substrate is set to n2, and a distance between the light reception element and the light shielding layer is set to h, the following Expression is satisfied.
Arctan((p-a/2-d/2)/h)≧Arcsin(n1/n2)

7. The bio-information acquisition device according to claim 6,
wherein an adhesion layer is included between the imaging section and the light shielding section, and
wherein a refractive index n3 of the adhesion layer is approximately equal to the refractive index n2 of the substrate.

8. The bio-information acquisition device according to claim 6, further comprising:
a light condensing section that includes a condensing lens which is disposed on an optical axis, in which the light reception element is connected with the opening section, between the light emitting section and the light shielding section,
wherein the light transmitting layer is provided between the light shielding section and the light condensing section.

9. The bio-information acquisition device according to claim 6,
wherein the light transmitting layer is a vacuum layer or an air layer.

10. The bio-information acquisition device according to claim 6,
wherein the light emitting element includes a reflecting layer that has a light reflection property, an electrode that has a light transmission property, and a light-emitting function layer that is disposed between the reflecting layer and the electrode,
wherein the light emitting section includes an insulating layer that is disposed between the reflecting layer and the electrode and decides a light emitting region in the light-emitting function layer, and a light transmitting section that is disposed between the adjacent light emitting elements, and
wherein an outer edge of the reflecting layer is located on a side of the light transmitting section rather than an end of the insulating layer on the side of the light transmitting section.

11. An electronic apparatus comprising the image acquisition device according to claim 1.

12. An electronic apparatus comprising the bio-information acquisition device according to claim 6.
